(19) United States
(12) Patent Application Publication
(10) Pub. No.: US 2024/0074891 A1
(43) Pub. Date: Mar. 7, 2024

(54) Mobile, External, Prophylactic, Female, Urinary System

(71) Applicant: Sandra Kay Click, Huntington Beach, CA (US)

(72) Inventor: Sandra Kay Click, Huntington Beach, CA (US)

(21) Appl. No.: 17/903,005

(22) Filed: Sep. 5, 2022

(51) Int. Cl. A61F 5/455; A61F 5/44

(52) U.S. Cl. CPC A61F 5/4556; A61F 5/4404

(57) ABSTRACT

The Mobile, External, Prophylactic, Female, Urinary System is a uniquely-configured system which allows a human female to void their bladder without urinary frustration or bladder discomfort—whether lying, sitting, standing, walking or traveling. This system contains uniquely-sized and uniquely-shaped components which capture a discharged stream of urine from a human female urethra, and direct it smoothly into a capture-and-storage bag. The system is worn external to the body, which helps preclude urinary-tract infection. It can be worn under clothing. It is useful when the user has: urinary incontinence, or difficulty getting to a toilet, or difficulty finding a convenient place to urinate while traveling.

### FIELD

This application relates to the field of medicine—specifically to the human female urinary tract; human female urinary-related catheters; and external, human female, urinary-related devices.

### BACKGROUND

Human female urinary catheters are generally used by females suffering from bladder incontinence and by females who are bed-ridden (or house-bound) due to health problems. Also, catheters are worn by those who find it extremely difficult to walk quickly and comfortably to the toilet. Additionally, catheters are also used when recent surgery causes immobility or very difficult mobility. Occasionally, catheters are used to permit traveling long distances without stopping the vehicle to use the toilet.

Most female urinary catheters are placed into (inside) the human female body through the urethral orifice. Sadly, there is a troublingly-high rate of infection; because catheters penetrate the body and provide an easy path for the entry of bacteria, germs and other infectious organisms.

Products have been developed as external (prophylactic) female urinary catheters, but many of these suffer from the drawback that the user is forced to remain motionless for the catheter device to function properly. Whether lying, sitting, or riding in a vehicle, remaining motionless for an extended duration is difficult, if not impossible, for most people.

Additionally, some existing external female catheters require a powered vacuum device which is cumbersome. Sometimes, an adequate vacuum-pump power source is not readily available.

Additionally, if the urinary stream emitted by the user is of significant volume, some existing external female catheters may have difficulty accommodating that significant volume.

### SUMMARY

This new MOBILE, EXTERNAL, PROPHYLACTIC, FEMALE, URINARY SYSTEM (hereafter: “Female Urinary System”) captures a discharged stream of urine from a human female urethra, and directs it smoothly through related components, and into a storage section, whether the user is lying, sitting, standing, or walking.

The Female Urinary System offers mobility, a “hands-free” mechanism, and a simpler system, which accommodates high volume urine flow.

Consequently, patient comfort, urine-control efficiency, and cleanliness is improved. Additionally, the probability of internal-catheter-related infection is reduced when compared to standard, body-inserted catheters.

The design is well suited for female children, who very frequently must interrupt the family vehicle trip to urinate.

The Female Urinary System may be used with, or without, underwear and/or clothing.

Economic advantages also occur because replacement components and maintenance components for the new Female Urinary System are simple and are relatively low-cost.

The Female Urinary System consists of several uniquely-configured, uniquely-shaped, specifically-tailored components which are uniquely-positioned and specifically-inter-connected. The intent of this application is to patent the entirety of the uniquely-sized, and uniquely shaped components, and the synergistically-integrated components as a complete system.

The Female Urinary System combines the functionality of a uniquely-sized, and uniquely shaped skin barrier (hereafter: “Skin Barrier”), a uniquely-sized, and uniquely shaped receiver coupler (hereafter: “Receiver Coupler”), a flexible bag—containing a receiver section, a urine transfer section, and a storage section—and a drain mechanism.

The depicted embodiment of the Female Urinary System shows a Flexible Bag, constructed from two, thin, flexible plastic sheets bonded or melded together at the edges, and also near the edges. Thus, the Flexible Bag is a water-tight, inner container surrounded by a flange-like “skirt”.

The depicted embodiment of the Female Urinary System employs two flexible, uniquely-sized, and uniquely shaped waterproof pads containing axially aligned holes.

The depicted embodiment of the Female Urinary System uses a commercially available, preexisting-component drain mechanism (IE: valve) which, no doubt, is individually patented and not claimed. However, the Female Urinary System is not required to use this pre-existing component, and various embodiments may have specialized, uniquely-manufactured components or, simply, a temporarily seal-able drain port.

The Female Urinary System may, optionally, employ a very low-level vacuum pump connected to a urine-trapping container, which attaches to a vacuum tube, which attaches to the drain mechanism. These vacuum-related components are pre-existing, covered by other patents, and not claimed or depicted.

An adhesive, waterproof, Skin Barrier is attached to the user's skin, and covers the major part of the human female labia majora, the vaginal orifice and the perineum (not including the anus).

This Skin Barrier has a hole (opening or void) slightly larger than the size of the user's urethral orifice. The Skin Barrier hole is positioned over, and aligned with, the urethral orifice.

A Receiver Coupler connects the Skin Barrier to the Flexible Bag at the Receiver Section. A hole through the Receiver Coupler closely matches the size of, and is axially aligned with, the hole in the Skin Barrier.

One side of the Flexible Bag, the side attached to the Receiver Coupler, has a hole which closely matches the size of, and is axially aligned with, the hole in the Receiver Coupler.

Consequently, urine may freely flow from the user, through the Skin Barrier, through the Receiver Coupler, and into the Flexible Bag. Urine is temporarily captured in the Receiver Section, but is free to immediately drain through a Urine Transfer Channel Section of the Flexible Bag into a larger Storage Section.

The Flexible Bag is held in the desired position (generally attached to one of the user's legs) with straps (covered by other patents and not depicted).

The Storage Section has a drainage mechanism used to ultimately dispose of the collected urine on demand.

### BRIEF DESCRIPTION OF THE DRAWINGS

FIG. 1 Is an exploded diagram of the Female Urinary System, showing the various components and unique features. Note: FIG. 1 is not necessarily to scale, but simply representative of the Female Urinary System components.

### DETAILED DESCRIPTION

The depicted disclosure describes a Female Urinary System which captures and transfers urine from the human female urethra to a Storage Section.

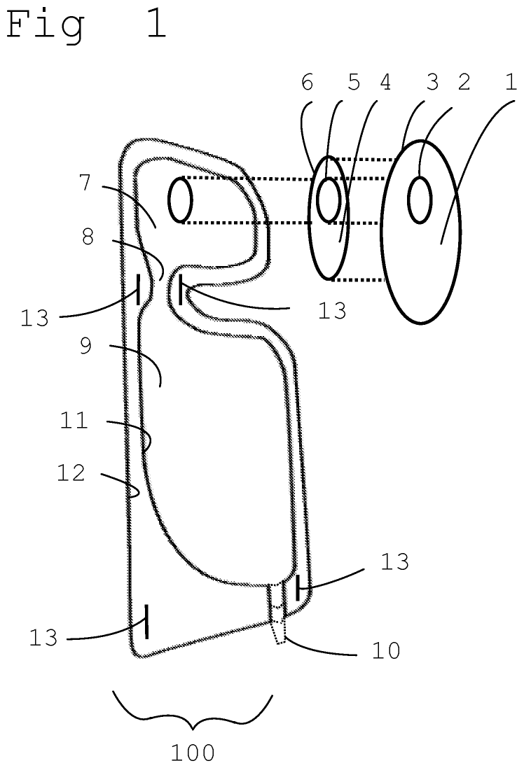

While describing the embodiment depicted in FIG. 1, it should be understood that urine from the body is flowing initially from right to left and then from top to bottom of FIG. 1.

With reference to FIG. 1A uniquely-sized and uniquely-shaped, soft, flexible, waterproof Skin Barrier 1 is affixed to the skin of a human female using a commercially-available, suitable adhesive. That adhesive is not claimed in this application. The Skin Barrier 1 has a Skin Barrier Outer Rim 2, which covers the human female labia majora and, generally, but not necessarily, the perineum. The Skin Barrier Outer Rim does not cover the Anus.

The Skin Barrier 1 has a specifically-placed and specifically-sized hole (void), defined by the Skin Barrier Inner Rim 3, slightly larger than, axially aligned with, and positioned over, the female urethral orifice. This hole permits urine from the female to pass through the Skin Barrier 1.

Attached by an adhesive to the Skin Barrier 1 is a uniquely-sized and uniquely-shaped, soft, flexible, waterproof Receiver Coupler 4. The Receiver Coupler 4 has a Receiver Coupler Inner Rim 5 which forms a hole (void) slightly larger than the hole of the Skin Barrier Inner Rim 3. The hole formed by the Receiver Coupler Inner Rim 5 is axially aligned with, and positioned over, the female urethral orifice. This larger size and orientation ensures unobstructed flow of discharged urine through the Receiver Coupler. The Receiver Coupler Outer Rim 6 is smaller than the Skin Barrier Outer Rim 2.

The Receiver Coupler 4 is bonded, or melded (by any commercially practical method), to a soft, flexible, waterproof Flexible Bag 100 at the Receiver Section 7 such that the the hole in the Receiver Coupler 4 axially aligns with the hole in the Receiver Section 7 of the Flexible Bag 100. The Receiver Section 7 may be of various size and/or shape.

In the depicted embodiment, the Receiver Section 7 opens to a Urine Transfer Channel Section 8. In other embodiments, this Urine Transfer Channel Section 8 may, or may not, have a one-way valve to prevent back-flow of urine toward the Urethra, and may be of various widths and lengths.

In the depicted embodiment, the Urine Transfer Channel Section 8 opens to a Storage Section 9. This Storage Section 9 is where the urine is collected and stored until the user is ready to empty the urine. This Storage Section 9 may be of various size and/or shape; providing it is sufficiently sized to accommodate the volume of urine discharged before convenient emptying.

In the depicted embodiment, the end of the Storage Section 9 is bonded or melded to a Drain Mechanism 10 which is used to dispose of collected urine on demand.

The Flexible Bag 100 may be constructed by adhesive bonding, or thermal or ultrasonic melting together of two thin, flexible plastic sheets along the Flexible Bag Inner Perimeter 11 and the Flexible Bag Outer Perimeter 12. The portion of the Flexible Bag 100 which expands is defined by the Flexible Bag Inner Perimeter 12. Alternatively, the Flexible Bag 100 could be constructed using a single, folded sheet of plastic, similarly bonded or melded to form the Flexible Bag 100 perimeters.

The “skirt”, formed between the Inner Perimeter 11 and the Outer Perimeter 12, has Leg Strap Slits 13 cut to accommodate commercially-available straps (not shown and not claimed) which hold the Flexible Bag 100 to the user's leg.

1. I claim a specifically-inter-connected, comprehensive, Mobile, External, Prophylactic, Female, Urinary System comprised of uniquely-sized, uniquely-configured, uniquely-shaped, specifically-tailored, and uniquely-positioned components, as defined in the specification and drawing, which captures a discharged stream of urine from a human female urethra, and directs it smoothly through related components, and into a capture-and-storage bag, whether the user is lying, sitting, standing or walking.
